(19) United States
(12) Patent Application Publication
(10) Pub. No.: US 2013/0102635 A1
(43) Pub. Date: Apr. 25, 2013

(54) Methods for Treating Disorders Associated with Hyperlipidemia in a Mammal

(71) Applicant: Gerald L. Wisler, Gladstone, NJ (US)

(72) Inventor: Gerald L. Wisler, Gladstone, NJ (US)

(21) Appl. No.: 13/444,519

(22) Filed: Apr. 11, 2012

Related U.S. Application Data
(63) Continuation of application No. 12/976,002, filed on Dec. 22, 2010, now abandoned.
Application No. 12/976,002 is a continuation of application No. 11/582,833, filed on Oct. 18, 2006, now abandoned.
(60) Provisional application No. 60/727,664, filed on Oct. 18, 2005.
(60) Provisional application No. 60/788,616, filed on Apr. 3, 2006.

(51) Int. Cl. A61K 31/4468; A61K 31/40

(52) U.S. Cl. CPC A61K 31/4468; A61K 31/40
USPC 514/325

(57) ABSTRACT

The invention is directed to methods for treating hyperlipidemia in a mammal. The methods involve combination therapies using a microsomal triglyceride transfer protein (MTP) inhibitor (for example, BMS-201038 and implitapide) and a HMG-CoA reductase inhibitor (for example simvastatin or atorvastatin). Co-administration of the MTP inhibitor with the HMG-CoA reductase inhibitor produces a therapeutic benefit, for example, a reduction in the concentration of cholesterol and/or triglycerides in the blood stream, but with fewer or reduced side effects than when higher dosages of the MTP inhibitor are used during monotherapy to provide the same or similar therapeutic benefit.

### RELATED APPLICATIONS

This application is a continuation of U.S. Ser. No. 11/582,833, filed Oct. 18, 2006, which claims the benefit of U.S. Provisional Patent Application Ser. No. 60/788,616, filed Apr. 3, 2006, and U.S. Provisional Patent Application Ser. No. 60/727,664, filed Oct. 18, 2005, the entire disclosures of which are incorporated by reference herein.

### FIELD OF THE INVENTION

This invention relates generally to methods of reducing the concentration of cholesterol and/or triglycerides in the blood of a mammal. More particularly, the invention relates to combination therapies using a microsomal triglyceride transfer protein (MTP) inhibitor and a HMG-CoA reductase inhibitor for reducing the concentration of cholesterol and/or triglycerides in the blood but with a reduced adverse event profile relative to MTP inhibitor monotherapy.

### BACKGROUND OF THE INVENTION

There are several known risk factors for atherosclerotic cardiovascular disease (ASCVD), the major cause of mortality in the Western world. One key risk factor is hyperlipidemia, which is the presence of elevated levels of lipids in blood plasma. Various epidemiological studies have demonstrated that drug mediated lowering of total cholesterol (TC) and low density lipoprotein (LDL) cholesterol (LDL-C) is associated with a significant reduction in cardiovascular events. The National Cholesterol Education Program's (NCEP's) updated guidelines recommends that the overall goal for high-risk patients is to achieve less than 100 mg/dL of LDL, with a therapeutic option to set the goal for such patients to achieve a LDL level less than 70 mg/dL.

One form of hyperlipidemia is known as hypertriglyceridemia and results in the presence of elevated amounts of triglycerides in the blood. Although triglycerides are necessary for good health, higher-than-normal triglyceride levels, often are associated with known risk factors for heart disease.

Another form of hyperlipidemia, known as hypercholesterolemia, which is the presence of elevated amounts of cholesterol in the blood, is a polygenic disorder. Modifications in lifestyle and conventional drug treatment are usually successful in reducing cholesterol levels. However, in some cases, as in familial hypercholesterolemia (FH), the cause is a monogenic defect. Treatment of a patient with FH can be more challenging because the levels of LDL-C remain elevated despite aggressive use of conventional therapy.

For example, one type of FH, homozygous familial hypercholesterolemia (hoFH), is a serious life-threatening genetic disease caused by homozygosity or compound heterozygosity for mutations in the low density lipoprotein (LDL) receptor. Patients with hoFH typically have total plasma cholesterol levels over 400 mg/dL resulting in premature atherosclerotic vascular disease. When left untreated, most patients develop atherosclerosis before age 20 and generally do not survive past age 30. However, patients diagnosed with hoFH are largely unresponsive to conventional drug therapy and have limited treatment options. Specifically, treatment with statins, which reduce LDL-C by inhibiting cholesterol synthesis and upregulating the hepatic LDL receptor, have negligible effect in patients whose LDL receptors are non-existent or defective. A mean LDL-C reduction of only less than about 20% has been recently reported in patients with genotype-confirmed hoFH treated with the maximal dose of statins (atorvastatin or simvastatin administered at 80 mg/day). The addition of ezetimibe 10 mg/day to this regimen resulted in a total reduction of LDL-C levels of 27%, which is still far from optimal. Non-pharmacological options have also been tested, including surgical interventions, such as portacaval shunt and ileal bypass, and orthotopic liver transplantation, but with clear disadvantages and risks. Therefore, there is a tremendous unmet medical need for new medical therapies for hoFH.

Microsomal triglyceride transfer protein (MTP) inhibitors have been developed as potent inhibitors of MTP-mediated neutral lipid transfer activity. MTP catalyzes the transport of triglyceride, cholesteryl ester, and phosphatidylcholine between small unilamellar vesicles. One exemplary MTP inhibitor is BMS-201038, developed by Bristol-Myers Squibb. See, U.S. Pat. Nos. 5,739,135; and 5,712,279. Studies using an animal model for homozygous FH indicated that BMS-201038 effectively reduced plasma cholesterol levels in a dose dependent manner, for example, at 25 mg/day, suggesting that this compound might be effective for treating patients with hoFH. It was noticed, however, that certain patients treated with 25 mg/day of BMS-201038 experienced certain adverse events, for example, gastrointestinal disturbances, abnormalities in liver function, and hepatic steatosis. Although a promising therapeutic agent, large scale clinical trials of BMS-201038 have been discontinued. Another potent MTP inhibitor known as implitapide has been developed. See, U.S. Pat. Nos. 6,265,431, 6,479,503, 5,952,498. During clinical studies, dosages of implitapide of 80 mg/day or greater, although therapeutically effective, were found to result in certain adverse events, for example, gastrointestinal disturbances, abnormalities in liver function, and hepatic steatosis. Large scale clinical studies using implitapide have also been discontinued.

Accordingly, there is still a need for methods for aggressively treating hyperlipidemias that effectively lower, for example, circulating cholesterol and triglycerides levels but with fewer or reduced adverse effects that typically result when higher dosages of the MTP inhibitor are used alone in monotherapy.

### SUMMARY OF THE INVENTION

The invention provides methods for lowering the concentration of cholesterol and/or triglycerides in the blood, and/or reducing the amount of one or more markers of atherosclerosis. The method includes administering a MTP inhibitor, such as, BMS-201038 or implitapide, in combination with a HMG-CoA reductase inhibitor, such as simvastatin or atorvastatin. The MTP inhibitors can be administered at certain lower dosages that are still therapeutically effective when combined with a HMG-CoA reductase inhibitor but yet create fewer or reduced adverse effects when compared to using therapeutically effective dosages of the MTP inhibitors during monotherapy.

In one aspect, the invention provides a method of reducing at least one of (i) the concentration of cholesterol and/or triglycerides in the blood of a mammal, and (ii) the amount of a marker of atherosclerosis in a mammal. The method comprises a combination therapy, which comprises administering each day to the mammal, for example, a human, a combination of HMG-CoA reductase inhibitor and BMS-201038, wherein BMS-201038 initially is administered at a first dosage in the range of 1 to 5 mg/day, for example, 2.5 mg/day or 5 mg/day, for at least 4 weeks, is then administered at a second dosage in the range of 3 to 7 mg/day, for example, 5 mg/day, for at least 4 weeks, and is then administered at a third dosage in the range of 6 to 9 mg/day, for example, 7.5 mg/day, for at least 4 weeks.

The method may include administering HMG-CoA reductase inhibitor at a dosage of 1 mg/day to 80 mg/day, more preferably at a dosage of 5 to 50 mg/day, and most preferably 10 mg/day to 20 mg/day. For example, the HMG-CoA reductase inhibitor may be administered at 5 mg/day, 10 mg/day, 20 mg/day, 40 mg/day or 80 mg/day or more. In one embodiment, a HMG-CoA reductase inhibitor is administered at a dosage of 10 mg/day. The HMG-CoA reductase inhibitor and BMS-201038 can be administered together in the same dosage form, or they may be administered in separate dosage forms. In the case of the separate dosage forms, HMG-CoA reductase inhibitor can be administered before, after, or simultaneously with BMS-201038.

The foregoing method may reduce the concentration of at least one of cholesterol and triglycerides in the blood but with a reduced incidence of an adverse event as compared to administration of a dosage of 25 mg/day of BMS-201038 during monotherapy. In another embodiment, the method reduces the number and/or amount of plaques, for example, arterial plaques, on a wall of a blood vessel of the mammal but with a reduced incidence of an adverse event as compared to administration of a dosage of 25 mg/day of BMS-201038 during monotherapy. Contemplated adverse events include, for example, gastrointestinal disturbances, abnormal liver function, and/or hepatic steatosis.

In another aspect, the invention provides a method of reducing at least one of (i) the concentration of cholesterol and/or triglycerides in the blood of a mammal, and (ii) the amount of a marker of atherosclerosis in a mammal. The method comprises administering each day to the mammal a combination of HMG-CoA reductase inhibitor and implitapide, wherein the implitapide is administered at a dosage in the range of 0.01 to 60 mg/day. It is understood that the implitapide preferably is administered at a dosage in the range of 20 to 60 mg/day, for example, 20 mg/day, 25 mg/day, 30 mg/day, 35 mg/day, 40 mg/day, 45 mg/day, 50 mg/day, 55 mg/day or even 60 mg/day or more.

Furthermore, it is understood, that the HMG-CoA reductase inhibitor is administered at a dosage of 1 to 100 mg/day, optionally, 1 to 80 mg/day, optionally 5 to 50 mg/day. For example, a HMG-CoA reductase inhibitor is administered at a dosage of 5 mg/day, 10 mg/day, 20 mg/day, 40 mg/day or 80 mg/day or more. The HMG-CoA reductase inhibitor and implitapide can be administered together in the same dosage form, or they may be administered in separate dosage forms. In the case of the separate dosage forms, HMG-CoA reductase inhibitor can be administered before, after, or simultaneously with implitapide.

This method may reduce the concentration of at least one of cholesterol or triglycerides in the blood but with a reduced incidence of an adverse event as compared to administration of a dosage of 80 mg/day or greater, e.g., as compared to 80 mg/day or 160 mg/day of implitapide during monotherapy. In another embodiment, the method reduces the amount of plaques, for example, arterial plaques, on a wall of a blood vessel of the mammal but with a reduced incidence of an adverse event as compared to administration of a dosage of 80 mg/day or greater, e.g., as compared to 80 mg/day or 160 mg/day of implitapide during monotherapy. Contemplated adverse events include, for example, gastrointestinial disorders, abnormalities in liver function, and/or hepatic steatosis.

The foregoing methods can be used to treat (i) patients with hyperlipidemia, for example, hypercholesterolemia (for example, homozygous or heterozygous familial hypercholesterolemia) or hypertriglyceridemia, (ii) patients resistant to statin monotherapy, (iii) statin-intolerant patients, and/or (iv) patients having a combination of (i) and (ii), (i) and (iii), (ii) and (iii), and (i), (ii) and (iii).

### DETAILED DESCRIPTION

This invention relates, in part, to methods of reducing at least one of (i) the concentration of cholesterol and/or triglycerides in the blood of a mammal, and (ii) the amount of a marker of atherosclerosis in a mammal. The methods are based on combination therapies where an MTP inhibitor, for example, BMS-201038 or implitapide, is administered with a HMG-CoA reductase inhibitor, for example, simvastatin or atorvastatin. The disclosed methods use lower dosages of the MTP inhibitor but, which in combination with the HMG-CoA reductase inhibitor, can be effective at reducing the concentration of cholesterol and/or triglycerides in the blood but with fewer adverse events, less severe adverse events and/or reduced frequency of adverse events resulting from the use of higher dosages of the MTP inhibitor during monotherapy.

In addition, the invention relates, in part, to a method of reducing gastrointestinal disorders or hepatic steatosis induced by BMS-201038 by administering BMS-201038 together with a HMG-CoA reductase inhibitor. Under certain circumstances, this approach may be useful at mitigating hepatic steatosis when dosages of BMS-201038 of 25 mg/day or greater are administered to the patient.

### 1. DEFINITIONS

For convenience, certain terms used in the specification, examples, and appended claims are collected in this section.

The phrase “combination therapy,” as used herein, refers to co-administering an MTP inhibitor, for example, BMS-201038 and implitapide, or a combination thereof, and HMG-CoA reductase inhibitor, for example, simvastatin or atorvastatin, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

The terms, “individual,” “patient,” or “subject” are used interchangeably herein and include any mammal, including animals, for example, primates, for example, humans, and other animals, for example, dogs, cats, swine, cattle, sheep, and horses. The compounds of the invention can be administered to a mammal, such as a human, but can also be other mammals, for example, an animal in need of veterinary treatment, for example, domestic animals (for example, dogs, cats, and the like), farm animals (for example, cows, sheep, pigs, horses, and the like) and laboratory animals (for example, rats, mice, guinea pigs, and the like).

The term, “patient resistant to statin monotherapy,” as used herein includes those patients for whom conventional statin monotherapy has been found ineffective or less effective than desired. A physician designing lipid reduction therapy for a patient will be able to determine via diagnosis and observation of periodic blood cholesterol and/or triglyceride levels whether such a patient is or has been resistant to statin monotherapy.

The term, “statin-intolerant patient,” as used herein includes those patients for whom conventional statin therapy, for example, for serum lipid reduction, has been found to be ineffective and/or for whom an effective lipid-reducing dose of statins is too high to be tolerated or that there is an unacceptable adverse event associated with a particular dose. For example, statin therapy may be discontinued by the physician/patient due to concern over an adverse event such as Liver Function Test abnormality, muscle aches and pains or inflammation—myalgia or myostitis, elevation in enzymes (CK) showing muscle adverse event. A physician designing lipid reduction therapy for a patient will be able to determine via diagnosis and observation of periodic blood cholesterol and/or triglyceride levels whether such a patient is statin-intolerant.

The phrase “minimizing adverse effects,” “reducing adverse events,” or “reduced adverse events,” as used herein refer to an amelioration or elimination of one or more undesired side effects associated with the use of MTP inhibitors of the present invention. Side effects of traditional use of the MTP inhibitors include, without limitation, nausea, gastrointestinal disorders, steatorrhea, abdominal cramping, distention, elevated liver function tests, fatty liver (hepatic steatosis); hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). Accordingly, the methods described herein provide an effective therapy while at the same time causing fewer or less significant adverse events.

In certain embodiments, side effects are partially eliminated. As used herein, the phrase “partially eliminated” refers to a reduction in the severity, extent, or duration of the particular side effect by at least 30%, 40%, 50%, 60%, 70%, 80%, 90%, 95% and 99% relative to that found by administering 25 mg/day of BMS-201038 during monotherapy or either 80 mg/day or 160 mg/day of implitapide during monotherapy. In certain embodiments, side effects are completely eliminated. Those skilled in the art are credited with the ability to detect and grade the severity, extent, or duration of side effects as well as the degree of amelioration of a side effect. In some embodiments, two or more side effects are ameliorated.

The term, “therapeutically effective” refers to the ability of an active ingredient, for example, BMS-201038 or implitapide, to elicit the biological or medical response that is being sought by a researcher, veterinarian, medical doctor or other clinician. Non-limiting examples include reduction of cholesterol (for example, LDL-C) and/or triglyceride levels in a patient, reduction of the amount of plaques, for example, arterial plaques, on the wall of a blood vessel, and the like.

The term, “therapeutically effective amount” includes the amount of an active ingredient, for example, BMS-201038, or implitapide, that will elicit the biological or medical response that is being sought by the researcher, veterinarian, medical doctor or other clinician. The compounds of the invention are administered in amounts effective at lowering the cholesterol concentration in the blood, and/or the triglyceride concentration in the blood and/or reducing the amount of plaques, for example, arterial plaques disposed upon the blood contacting wall of one or more blood vessels. Alternatively, a therapeutically effective amount of an active ingredient is the quantity of the compound required to achieve a desired therapeutic and/or prophylactic effect, such as the amount of the active ingredient that results in the prevention of or a decrease in the symptoms associated with the condition.

The terms, “pharmaceutically acceptable” or “pharmacologically acceptable” refer to molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein, the phrase, “BMS-201038” refers to a compound known as N-(2,2,2-Trifluorethyl)-9-[4-[4-[[[4′-(trifluoromethyl)[1,1′biphenyl]-2-Yl]carbonyl]amino]-1-piperidinyl]butyl] 9H-fluorene-9-carboxamide, having the formula:

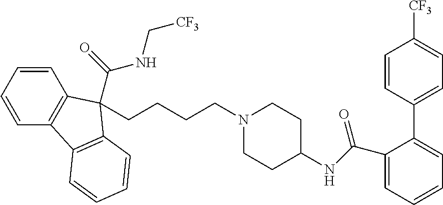 [SMILES: O=C(NC1CCN(CCCCC2(C(=O)NCC(F)(F)F)c3ccccc3-c3ccccc32)CC1)c1ccccc1-c1ccc(C(F)(F)F)cc1]

stereoisomers thereof, and/or pharmaceutically acceptable salts or esters thereof.

As used herein, the phrase “implitapide” refers to a compound known as (2S)-2-cyclopentyl-2-[4-[(2,4-dimethyl-9H-pyrido[2,3-b]indol-9-yl)methyl]phenyl]-N-[(1S)-2-hydroxy-1-phenylethyl]ethanamide and having the structure shown below:

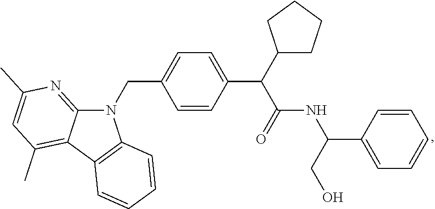 [SMILES: Cc1cc(C)c2c3ccccc3n(Cc3ccc(C(C(=O)NC(CO)c4ccccc4)C4CCCC4)cc3)c2n1]

stereoisomers thereof, and/or pharmaceutically acceptable salts or esters thereof.

As used herein “HMG-CoA reductase inhibitors,” commonly referred to as “statins” refer to agents that slow down the body's production of cholesterol and inhibit the bioconversion of hydroxymethylglutaryl-coenzyme A to mevalonic acid catalyzed by the enzyme HMG-CoA reductase. Exemplary statins can include atorvastatin (7-[2-(4-fluorophenyl)-5-(1-methylethyl)-3-phenyl-4-(phenylcarbamoyl)-1H-pyrrol-1-yl]-3,5-dihydroxy-heptanoic acid; brand name: Lipitor), fluvastatin (sodium 7-[3-(4-fluorophenyl)-1-propan-2-yl-indol-2-yl]-3,5-dihydroxy-hept-6-enoate; brand name: Lescol), lovastatin (8-[2-(4-hydroxy-6-oxo-tetrahydropyran-2-yl)ethyl]-3,7-dimethyl-1,2,3,7,8,8a-hexahydronaphthalen-1-yl] 2-methylbutanoate; brand names: Altocor, Mevacor), pravastatin (5-dihydroxy-7-[6-hydroxy-2-methyl-8-(2-methylbutanoyloxy)-1,2,6,7,8,8a-hexahydronaphthalen-1-yl]-heptanoicacid; brand name: Pravachol), rosuvastatin (7-[4-(4-fluorophenyl)-6-(1-methylethyl)-2-(methyl-methylsulfonylamino)-pyrimidin-5-yl]-3,5-dihydroxy-hept-6-enoic acid; brand name: Crestor), pitavastatin, tenivastatin, simvastatin (7-(2,6-dimethyl-8-(2,2-dimethylbutyryloxy)-1,2,6,7,8,8a-hexahydro-1-naphthyl)-3,5-dihydroxyheptanoic acid; brand name: Zocor), rivastatin, mevastatin, or cerivastatin. It is contemplated that each of the foregoing compounds also include stereoisomers of such compounds, and/or pharmaceutically acceptable salts or esters of such compounds.

Pharmaceutically acceptable salts of the foregoing compounds can be synthesized, for example, from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. The terms “racemate,” “racemic mixture” or “racemic modification” refer to a mixture of equal parts of enantiomers.

### 2. METHODS OF THE INVENTION

In general the invention provides methods for treating hyperlipidemia using one or more MTP inhibitors, for example, BMS-201038 or implitapide. The MTP inhibitors can be used at dosages lower than those already found to result in one or more adverse events, for example, gastrointestinal disorders, abnormalities in liver functional and/or hepatic steatosis (for example, 25 mg/day of BMS-201038, 80 mg/day of implitapide and 160 mg/day of implitapide have been found to cause gastrointestinal disorders, abnormalities in liver function and/or hepatic steatosis) but are still are therapeutically effective when combined with a HMG-CoA reductase inhibitor, for example, simvastatin or atorvastatin. It is contemplated that statins may be effective at reducing steatosis even when dosages of BMS-201038 of 25 mg/day or greater are administered.

(a) Combination Therapies Using BMS-201038 and a HMG-CoA Reductase Inhibitor

In certain aspects, the invention provides a method of reducing at least one of (i) the concentration of cholesterol and/or triglycerides in the blood of a mammal, and (ii) the amount of a marker of atherosclerosis in the blood stream of a mammal. The method comprises a combination therapy, which can be achieved by co-administering to the mammal, each day, HMG-CoA reductase inhibitor and BMS-201038. In one protocol, BMS-201038 initially is administered at a first dosage in the range of 1 to 5 mg/day for at least 4 weeks, is then administered at a second dosage in the range of 3 to 7 mg/day for at least 4 weeks, and is then administered at a third dosage in the range of 6 to 9 mg/day for at least 4 weeks.

The first dosage of BMS-201038 can be for example 2.5 mg/day or 5 mg/day. The second dosage of BMS-201038 can be 5 mg/day. The third dosage of BMS-201038 can be 7.5 mg/day. In certain embodiments, the second dosage is administered immediately following the first dosage, i.e., the second dosage is administered starting at five weeks from the initial first dosage. Similarly, in certain other embodiments, the third dosage of BMS-201038 is administered immediately following the second dosage, e.g., the second dosage is administered at nine weeks from the initial first dosage.

Optionally, the method may include administering a fourth dosage of BMS-201038 alone, or in combination with a HMG-CoA reductase inhibitor. Such a fourth dosage may be in the range of 9-12 mg/day, or more of BMS-201038. A fourth dosage may occur immediately after the third dosage, or may occur after a time interval, for example, a day, days, a week, or weeks after the third dosage. The fourth dosage may be administered to the subject for 1, 2, 3, 4 or more weeks.

In this approach, a HMG-CoA reductase inhibitor is co-administered at a dosage in the range of 0.01 to 100 mg/day, more preferably at a dosage in the range of 1 to 50 mg/day. For example, a HMG-CoA reductase inhibitor may be administered at a dosage of 10 mg/day. The HMG-CoA reductase inhibitor and BMS-201038 can be administered together in the same dosage form, or they may be administered in separate dosage forms. In the case of the separate dosage forms, a HMG-CoA reductase inhibitor can be administered before, after, or simultaneously with BMS-201038.

The methods disclosed herein may occur before or after other dosing regimens that may include, for example, BMS-201038 and/or other MTP inhibitors, a HMG-CoA reductase inhibitor, and/or other lipid-lowering agents. For example, the methods disclosed herein may occur after a patient has received ezetimibe monotherapy or ezetimibe combination therapy.

This approach may reduce the concentration of at least one of cholesterol or triglycerides in the blood but with a reduced incidence of an adverse event as compared to administration of a dosage of 25 mg/day of BMS-201038 during monotherapy. In addition or in the alternative, the method reduces the amount of plaques, for example, arterial plaques, on a wall of a blood vessel of the mammal but with a reduced incidence of an adverse event as compared to administration of a dosage of 25 mg/day of BMS-201038 during monotherapy. The amount of arterial plaques and the reduction thereof, can be measured using conventional non-invasive techniques known in the art, for example, magnetic resonance imaging, computerized tomography, and nuclear scintigraphic techniques. Contemplated adverse events include, for example, gastrointestinal abnormalities, hepatic steatosis, etc.

In certain other embodiments, the method produces an approximately 35%, 40% or more decrease in LDL-C in patients as compared to the patient's LDL-C level before treatment.

The methods disclosed herein may reduce or lower the concentration of serum cholesterol. It is understood that total serum cholesterol can be provided by very low density lipoproteins (VLDL), intermediate density lipoproteins (IDL), LDL and chylomicrons. Accordingly, it is contemplated that the combination therapies may reduce total blood cholesterol, or cholesterol provided by or associated with VLDL, IDL, LDL and chylomicrons. In addition, the methods disclosed herein may reduce or lower the concentration of serum triglycerides. It is understood that the serum triglycerides can be provided by VLDL and chylomicrons, and to a lesser extent by IDL and LDL. Accordingly, it is contemplated that the combination therapies may reduce triglycerides provided by or associated with VLDL, IDL, LDL and chylomicrons.

In some cases, the methods provided herein may reduce markers of atherosclerosis in the blood, such as, inflammatory markers (for example, c-reactive protein (CRP), interleukin-6 (IL-6), interleukin-1 (IL-1), CD-40, tissue necrosis factor-α (TNF-α), serum amyloid A, fibrinogen, urinary monocyte chemoattracant protein (MCP-1), neopterin, IL-1 receptor, IL-18, IL-10), oxidative markers (for example, myeloperoxidase (MPO), oxidized tyrosine residues, oxidized LDL (ox-LDL), lipoprotein-associated phospholipase A2 (Lp-PLA2), F2-isoprostanes, ox-LDL autoantibodies (IgG, IgM), and malondialdehyde (MDA)), endothelial markers (for example, intracellular adhesion molecule (ICAM), vascular cell adhesion molecule (VCAM), e-selectin, nitrate/nitrite), arterial remodeling markers (for example, matrix metalloproteinases (MMPs)/tissue inhibitors of MMPs (TIMPs), PICP, PINP, and/or platelet/thrombosis markers (e.g. p-selectin, tissue factor, heparin co-factor). An exemplary marker for atherosclerosis is CRP, which is a marker for inflammation that is believed to be a predictor of chronic heart disease. Decreases in serum cholesterol and/or triglyceride levels likely leads to a reduction in the build up of plaque, and may in some cases actually lead to regression in plaque.

In another aspect, the invention provides a method of reducing hepatic steatosis in a patient receiving BMS-201038. The method comprises co-administering BMS-201038 and a HMG-CoA reductase inhibitor to the patient. The BMS-201038 may be administered, for example, at a dosage from 1 to 10 mg/day, or 1 to 25 mg/day, or at a dosage greater than 10 mg/day, for example, 10 to 80 mg/day. Higher doses may be appropriate for HoFH or severe refractory patients. BMS-201038 and a HMG-CoA reductase inhibitor may be administered together in the same dosage form or may be administered in separate dosage forms. In the case of separate dosage forms, a HMG-CoA reductase inhibitor is administered before, after, or simultaneously with, BMS-201038.

(b) Combination Therapies Using Implitapide and a HMG-CoA Reductase Inhibitor

In another aspect, the invention provides a method of reducing at least one of (i) the concentration of cholesterol and/or triglycerides in the blood of a mammal, and (ii) the amount of a marker of atherosclerosis in a mammal. The method comprises a combination therapy wherein a combination of a HMG-CoA reductase inhibitor and implitapide are administered to the mammal each day.

It is understood that the implitapide is administered at a dosage in the range of 0.01 to 60 mg/day, more preferably in the range of 20 to 60 mg/day, for example, 20 mg/day, 25 mg/day, 30 mg/day, 35 mg/day, 40 mg/day or 60 mg/day. Furthermore, it is understood, that the a HMG-CoA reductase inhibitor is administered at a dosage of 0.01 to 100 mg/day, optionally, 1 to 50 mg/day, optionally 1 to 25 mg/day. For example, a HMG-CoA reductase inhibitor is administered at a dosage of 5 mg/day, 10 mg/day, 20 mg/day, 40 mg/day, or 85 mg/day. The HMG-CoA reductase inhibitor and implitapide can be administered together in the same dosage form, or they may be administered in separate dosage forms. When administered in separate dosage forms, the implitapide can be administered before, after, or simultaneously with, the a HMG-CoA reductase inhibitor.

The foregoing method may reduce the concentration of at least one of cholesterol or triglycerides in the blood but with a reduced incidence of an adverse event as compared to administration of a dosage of 80 mg/day or more, for example as 160 mg/day, of implitapide during monotherapy. In another embodiment, the method reduces the amount of plaques, for example, arterial plaques, on a wall of a blood vessel of the mammal but with a reduced incidence of an adverse event as compared to administration of a dosage of 80 mg/day or more, for example as 160 mg/day, of implitapide, during monotherapy. Contemplated adverse events include, for example, hepatic steatosis. Furthermore, this protocol may also reduce the presence and/or amount of one or more of the aforementioned markers of atherosclerosis.

### 3. FORMULATION AND ADMINISTRATION OF THE ACTIVE INGREDIENTS

In certain embodiments, the MTP inhibitor (for example, BMS-201038 and implitapide) and the HMG-CoA reductase inhibitor (for example, simvastatin or atorvastatin) are administrated orally. For oral administration, the active ingredients may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, talc, silica and the like), disintegrating agents (e.g. potato starch, sodium starch glycollate and the like), wetting agents (e.g. sodium laurylsulphate) and the like. Such tablets may also be coated by methods well known in the art.

Although less preferred, it is contemplated that the active ingredients may be formulated for, and administered by parenteral routes, for example, by intravenous routes, intramuscular routes, and by absorption through mucous membranes. It is contemplated that such formulations and parenteral modes of administration are known in the art.

The dosages described above may be administered in single or divided dosages of one to four times daily. The MTP inhibitor and HMG-CoA reductase inhibitor may be employed together in the same dosage form or in separate dosage forms taken at the same time, or at different times.

The methods described herein are particularly useful for treating patients, for example, LDL reduction-resistant patients, patients unable to achieve the cholesterol and/or LDL cholesterol goals desired by their physician and/or outlined by the NCEP guidelines. This inability may be due to an inability to tolerate an MTP inhibitor (e.g., BMS-201038 and implitapide) and/or a HMG-CoA reductase inhibitor (e.g., simvastatin or atorvastatin), or the inability of existing agents to provide sufficient cholesterol lowering to achieve these goals (for example, too much active ingredient is required achieve the desired end point). The methods described herein are especially useful for higher risk patients, for example, patients with coronary heart disease or with a similar risk of a coronary event. Such patients may have a 10 year risk of a coronary event of greater than 20%.

For example, the disclosed methods may be useful at treating LDL reduction-resistant patients, for example, patients with coronary heart disease or coronary heart disease risk equivalent patients with severe hypercholesterolemia of any etiology unable to come within 25%, more preferably 15%, of their NCEP LDL cholesterol goal on maximal tolerated oral therapy, as determined by their prescribing physician based upon established NCEP guidelines. Alternatively, in another preferred embodiment, the methods may be used for the treatment of severe hypercholesterolemia of any etiology unable to come within 75 mg/dL of NCEP LDL cholesterol goal on maximal tolerated oral therapy. The methods disclosed herein may include patients with severe hypertriglyceridemia unable to reduce total triglyceride levels (Tg) of <1000 or <500 mg/dL on maximal tolerated therapy.

In another embodiment, patients who have demonstrated intolerance to statins may be treated using the disclosed methods. For example, such methods may be effective for a statin intolerant patient, for example, where the therapy has been discontinued by the patient's physician and/or by the patient due to concern over an adverse event (for example, a liver function test abnormality, muscle aches and pains or inflammation such as myalgia or myostitis, and/or elevation in enzymes (CK) showing muscle adverse event).

In certain embodiments, the methods disclosed herein, may minimize at least one of side effects associated with the administration of BMS-201038 and/or implitapide. Such side effects include, for example, gastrointestinal disorders, nausea, steatorrhea, abdominal cramping, distention, elevated liver function tests such as increases in liver enzymes such as alanine, minor fatty liver, hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). In some embodiments the minimization of the side effect is determined by assessing the grade, severity, extent, or duration by subject questionnaire.

### EXAMPLES

The examples that follow are intended in no way to limit the scope of this invention but are provided to illustrate the methods present invention. Many other embodiments of this invention will be apparent to one skilled in the art.

### Example 1

### BMS-201038/HMG-CoA reductase inhibitor Combination Therapy

This study is designed to show that doses of BMS-201038 significantly lower than 25 mg/day, in combination with a HMG-CoA reductase inhibitor, can provide clinically significant reductions in LDL-C while still providing an improved adverse event profile. The primary parameter of efficacy in this study will be the percentage change in LDL-C after 16 weeks of therapy.

Approximately 175 subjects, both men and women, will be randomized into one of five treatment arms with equal probability. The subjects will have a baseline LDL of 130-250 mg/dL and baseline triglyceride level of less than 400 mg/dL. In treatment arm 1, subjects receive a placebo. In treatment arm 2, subjects receive BMS-201038 (2.5 mg) plus atorvastatin placebo. In effect, treatment arm 2 represents monotherapy with BMS-201038. In treatment arm 3, subjects receive 10 mg of atorvastatin, effectively representing monotherapy with atorvastatin. In treatment arm 4, subjects receive 10 mg atorvastatin plus ezetimibe (10 mg). In treatment arm 5, subjects receive BMS-201038 (2.5 mg) plus atorvastatin (10 mg). Patients in treatment arms 4 and 5 patients, in effect, receive a combination therapy.

After 4 weeks of treatment, subjects in arms 2 and 5 receive a step-up in concentration of BMS-201038 from 2.5 mg to 5 mg for 4 weeks. Thereafter, subjects in arms 2 and 5 then receive a second step-up in concentration in BMS-201038 from 5 mg to 7.5 mg for 4 more additional weeks of treatment. After another 4 weeks of treatment, subjects in arms 2 and 5 then receive a third step-up in concentration in BMS-201038 from 7.5 mg to 10 mg for 4 additional weeks of treatment. Subjects in arm 2 receive atorvastatin matching placebo for the entire 16 weeks of treatment. Subjects randomized to receive atorvastatin (10 mg/day) in arms 3 (alone), 4 (in combination with BMS-201038) and 5 (in combination with implitapide) remain on these doses of atorvastatin for the entire 16-week treatment period.

Throughout the study, blood samples are removed from each of the test patients for testing, for example, for testing the level of LDL-C, total cholesterol, triglycerides, HDL-C, Non-HDL-C, Apo B, and Apo A1 in each patient. Changes in body weight of the subjects are measured as part of vital signs collection.

### EQUIVALENTS

It is understood that the disclosed invention is not limited to the particular methodology, protocols, and dosages described as these may vary. It is also to be understood that the terminology used herein is for the purpose of describing particular embodiments only, and is not intended to limit the scope of the present invention which will be limited only by the appended claims.

### INCORPORATION BY REFERENCE

The entire disclosure of each of the patent documents and scientific articles referred to herein is incorporated by reference for all purposes.

What is claimed is:

1. A method of reducing at least one of (i) the concentration of cholesterol and/or triglycerides in the blood of a mammal, and (ii) the amount of a marker of atherosclerosis in a mammal, the method comprising administering each day to the mammal a combination of HMG-CoA reductase inhibitor and BMS-201038, wherein BMS-201038 initially is administered at a first dosage in the range of 1 to 5 mg/day for at least 4 weeks, is then administered at a second dosage in the range of 3 to 7 mg/day for at least 4 weeks, and is then administered at a third dosage in the range of 6 to 9 mg/day for at least 4 weeks.

2. The method of claim 1, further comprising administering, after the third dosage, a fourth dosage of BMS-201038 in the range of 9 to 12 mg/day for at least 4 weeks.

3. The method of claim 1, wherein the first dosage is 2.5 mg/day.

4. The method of claim 1, wherein the second dosage is 5 mg/day.

5. The method of claim 1, wherein the third dosage is 7.5 mg/day.

6. The method of claim 2, wherein the fourth dosage is 10 mg/day.

7. The method of claim 1, wherein the HMG-CoA reductase inhibitor is administered at a dosage of 1 to 80 mg/day.

8. The method of claim 7, wherein the HMG-CoA reductase inhibitor is administered at a dosage of 5 to 50 mg/day.

9. The method of claim 8, wherein the HMG-CoA reductase inhibitor is administered at a dosage of 10 to 20 mg/day.

10. The method of claim 1, wherein the HMG-CoA reductase inhibitor and BMS-201038 are administered together in the same dosage form.

11. The method of claim 1, wherein the HMG-CoA reductase inhibitor and BMS-201038 are administered in separate dosage forms.

12. The method of claim 1, wherein the HMG-CoA reductase inhibitor is mevastatin, lovastatin, pravastatin, simvastatin, fluvastatin, cerivastatin, atorvastatin, tenivastatin, rosuvastatin, and pitavastatin.

13. The method of claim 1, wherein the HMG-CoA reductase inhibitor is simvastatin or atorvastatin.

14. The method of claim 1, wherein the mammal is a human.

15. The method of claim 14 wherein the human is a patient resistant to statin monotherapy.

16. The method of claim 14 wherein the human is a statin-intolerant patient.

17. The method of claim 14 wherein the human has hyperlipidemia, hypercholesterolemia, hyperchylomicronemia, or a combination thereof.

18. The method of claim 17, wherein hypercholesterolemia is homozygous or heterozygous familial hypercholesterolemia.

19. The method of claim 14, wherein the method reduces the concentration of cholesterol or triglycerides in the blood but with a reduced incidence of an adverse event as compared to administration of a dosage of 25 mg/day of BMS-201038.

20. The method of claim 14, wherein the method reduces the amount of arterial plaques on a wall of a blood vessel of the mammal but with a reduced incidence of an adverse event as compared to administration of a dosage of 25 mg/kg of BMS-201038.

21. The method of claim 19 wherein the adverse event is hepatic steatosis.
